(19) United States
(12) Patent Application Publication
(10) Pub. No.: US 2018/0298065 A1
(43) Pub. Date: Oct. 18, 2018

(54) PROTEASE-RESISTANT STREPTAVIDIN

(71) Applicant: EUROPEAN MOLECULAR BIOLOGY LABORATORY, Heidelberg (DE)

(72) Inventors: Jeroen KRIJGSVELD, Epfenbach (DE); Mahmoud-Reza RAFIEE, Dossenheim (DE)

(21) Appl. No.: 15/762,079

(22) PCT Filed: Sep. 23, 2016
(86) PCT No.: PCT/EP2016/072633

§ 371 Date: Mar. 21, 2018

(30) Foreign Application Priority Data
Sep. 23, 2015 (EP) 15186564.9

(51) Int. Cl. C07K 14/36; G01N 33/53

(52) U.S. Cl. CPC C07K 14/36; G01N 2440/32; G01N 33/5308

(57) ABSTRACT

The present invention relates to modified streptavidin molecules that are resistant to cleavage by Lys-C or other proteases. These modified streptavidin molecules can be produced by chemical modification of natural streptavidin, by chemical synthesis or by genetic engineering. The invention also relates to nucleic acid molecules encoding these modified streptavidin molecules, to vectors comprising such nucleic acid molecules, and to cells comprising such nucleic acid molecules or vectors. The invention further relates to solid supports and kits comprising the modified streptavidin molecules. The invention also relates to the use of such modified streptavidin molecules or such solid supports for the capture/immobilization of proteins, peptides, oligonucleotides (e.g. aptamers), polynucleotides (e.g. DNA, RNA, or PNA), lipids, (poly) saccharides, carbohydrates, metabolites, drugs and small molecules, natural and synthetic molecules and to the use of these modified streptavidin molecules or these solid supports in mass spectrometry for the identification of proteins that interact with aforementioned (bio)molecules. The invention further relates to a method for reducing background in mass spectrometry by employing the modified streptavidin molecules.

### FIELD OF THE INVENTION

The present invention relates to modified streptavidin molecules that are resistant to cleavage by Lys-C or other proteases. These modified streptavidin molecules can be produced by chemical modification of natural streptavidin, by chemical synthesis or by genetic engineering. The invention also relates to nucleic acid molecules encoding these modified streptavidin molecules, to vectors comprising such nucleic acid molecules, and to cells comprising such nucleic acid molecules or vectors. The invention further relates to solid supports and kits comprising the modified streptavidin molecules. The invention also relates to the use of such modified streptavidin molecules or such solid supports for the capture/immobilization of proteins, peptides, oligonucleotides (e.g. aptamers), polynucleotides (DNA, RNA, or PNA), lipids, (poly)saccharides, carbohydrates, metabolites, drugs and small molecules, natural and synthetic molecules and to the use of these modified streptavidin molecules or these solid supports in mass spectrometry for the identification of proteins that interact with aforementioned (bio)molecules. The invention further relates to a method for reducing background in mass spectrometry by employing the modified streptavidin molecules.

### BACKGROUND OF THE INVENTION

Mass spectrometry is an established method in protein analysis. However, full-length proteins are often too large for mass spectroscopic analysis. Thus, full-length proteins are usually digested by proteases to obtain smaller fragments that are suitable for analysis.

The present inventors regularly use a protocol in which the target protein to be analysed is tagged with biotin. The biotinylated protein is purified by affinity chromatography using the strong binding affinity between biotin and streptavidin. This is achieved by using an affinity column with a support material to which streptavidin is covalently attached or by using beads to which streptavidin is covalently attached. To generate protein fragments that are suitable for mass spectroscopic analysis, a protease (e.g. LysC or trypsin) is directly applied to the affinity column or to the beads when the biotinylated target protein is bound to the streptavidin, or after elution of biotinylated proteins off such beads/support.

However, the proteolytic digestion does not only cleave the target protein but also the streptavidin covalently bound the support material or to the beads. The proteolytic fragments of streptavidin form an undesired background in the subsequent mass spectroscopic analysis.

Even when the protein is eluted from the beads/support prior to the protease treatment, often a background of streptavidin fragments is observed, since leakage of streptavidin from the beads/support occurs despite the covalent linkage. Presumably, leakage occurs because streptavidin is a tetramer of which only one subunit is covalently attached to the beads or the support material.

The inventors also use an experimental protocol in which proteins interacting with a molecule of interest are identified. To this end, the molecule of interest is tagged with biotin and bound to a support material or beads to which streptavidin is covalently attached. A sample containing a potential interaction partner is then contacted with the support material or the beads so that the potential protein interaction partner is captured. The protein interaction can then be subjected to proteolytic digestion (optionally after elution off the support material or off the beads) and the proteolytic fragments can be analysed by mass spectrometry. Again an undesired background of streptavidin fragments is often observed, regardless whether proteolytic digestion is carried out on the beads/support or after elution of the protein interaction partner.

### TECHNICAL PROBLEMS UNDERLYING THE PRESENT INVENTION

The present inventors have now found a way to prepare modified streptavidin molecules that are resistant to cleavage by proteases (in particular resistant to cleavage by LysC and/or tryp sin) while still maintaining a high binding affinity to biotin.

The novel modified streptavidins of the instant invention are well-suited for applications in protein purification, especially for subsequent protein identification by mass spectrometry. However, the novel modified streptavidins can advantageously be used in other methods that need stable (i.e. protease-resistant) streptavidins with high binding affinity to biotin.

The above overview does not necessarily describe all problems solved by the present invention.

### SUMMARY OF THE INVENTION

In a first aspect the present invention relates to a modified streptavidin that (i) is resistant to cleavage by at least one endopeptidase, wherein said at least one endopeptidase is specific for a basic amino acid; and (ii) exhibits a dissociation constant KD to biotin of 10⁻¹⁰ M or less.

In a second aspect the present invention relates to a nucleic acid molecule comprising a nucleotide sequence which encodes the modified streptavidin according to the first aspect.

In a third aspect the present invention relates to a vector comprising the nucleic acid molecule according to the second aspect. In a fourth aspect the present invention relates to a cell comprising the nucleic acid molecule of the second aspect or the vector of the third aspect.

In a fifth aspect the present invention relates to a solid support comprising the modified streptavidin of the first aspect.

In a sixth aspect the present invention relates to a kit comprising the modified streptavidin of the first aspect or the solid support of the fifth aspect and further comprising at least one protease selected from the group consisting of LysC, LysN, ArgC, and trypsin.

In a seventh aspect the present invention relates to a use of the modified streptavidin of the first aspect or the solid support of the fifth aspect or the kit of the sixth aspect for capture or immobilization of at least one biotinylated molecule. In an eighth aspect the present invention relates to a use of the modified streptavidin of the first aspect or the solid support of the fifth aspect or the kit of the sixth aspect for protein purification.

In a ninth aspect the present invention relates to a use of the modified streptavidin of the first aspect or the solid support of the fifth aspect or the kit of the sixth aspect in mass spectroscopy.

In a tenth aspect the present invention relates to a method for reducing background in mass spectrometry, comprising the steps:
(i) providing beads carrying the modified streptavidin according to the first aspect;
(ii) contacting a sample comprising a biotinylated protein with the beads of step (i), thereby binding the biotinylated protein to the modified streptavidin;
(iii) optionally washing the beads with a wash buffer;
(iv) adding a solution comprising a protease to the beads, thereby generating peptide fragments of the biotinylated protein;
(v) recovering the peptide fragments generated in step (iv); and
(vi) optionally subjecting the peptide fragments recovered in step (v) to mass spectrometric analysis.

In an eleventh aspect the present invention relates to a method for reducing background in mass spectrometry, comprising the steps:
(i) providing beads carrying the modified streptavidin according to the first aspect;
(ii) contacting a sample comprising a biotinylated protein with the beads of step (i), thereby binding the biotinylated protein to the modified streptavidin;
(iii) optionally washing the beads with a wash buffer;
(iv) eluting the biotinylated protein;
(v) adding a solution comprising a protease to the biotinylated protein eluted in step (iv), thereby generating peptide fragments of the biotinylated protein;
(vi) recovering the peptide fragments generated in step (v); and
(vii) optionally subjecting the peptide fragments recovered in step (vi) to mass spectroscopic analysis.

In a twelfth aspect the present invention relates to a method for capturing a protein interaction partner of a molecule, comprising the steps:
(i) providing beads carrying the modified streptavidin according to the first aspect;
(ii) contacting a biotinylated molecule with the beads of step (i), thereby loading the biotinylated molecule onto the beads;
(iii) contacting a sample with the beads loaded with the biotinylated molecule obtained in step (ii), wherein said sample comprises at least one protein interaction partner for the biotinylated molecule;
(iv) optionally washing the beads with a wash buffer;
(v) adding a solution comprising a protease to the beads, thereby generating peptide fragments of the at least one protein interaction partner;
(vi) recovering the peptide fragments generated in step (v); and
(vii) optionally subjecting the peptide fragments recovered in step (v) to mass spectroscopic analysis.

In a thirteenth aspect the present invention relates to a method for capturing a protein interaction partner of a molecule, comprising the steps:
(i) providing beads carrying the modified streptavidin according to the first aspect;
(ii) contacting a biotinylated molecule with the beads of step (i), thereby loading the biotinylated molecule onto the beads;
(iii) contacting a sample with the beads loaded with the biotinylated molecule obtained in step (ii), wherein said sample comprises at least one protein interaction partner for the biotinylated molecule;
(iv) optionally washing the beads with a wash buffer;
(v) eluting the at least one protein interaction partner;
(vi) adding a solution comprising a protease to the protein interaction partner eluted in step (v), thereby generating peptide fragments of the at least one protein interaction partner;
(vii) recovering the peptide fragments generated in step (vi); and
(viii) optionally subjecting the peptide fragments recovered in step (vii) to mass spectroscopic analysis.

In a fourteenth aspect the present invention relates to a method for capturing chromatin-associated proteins, comprising the steps:
(i) providing cells the chromatin of which is to be investigated;
(ii) adding formaldehyde to the cells to crosslink chromatin;
(iii) shearing the chromatin sample, thereby generating a sheared chromatin sample;
(iv) adding an antibody that is specific for a chromatin-associated protein of interest to the cross-linked and sheared chromatin-sample of step (iii), thereby immuno-precipitating the protein of interest and molecules cross-linked to the protein of interest;
(v) contacting the immuno-precipitated protein from step (iv) with beads coated with protein A or protein G, thereby immobilizing the immuno-precipitated protein on the beads;
(vi) optionally washing the beads with a wash buffer;
(vii) adding biotinylated nucleotides and a DNA polymerase to the immuno-precipitated protein of step (v) or, when present, of step (vi), thereby biotinylating DNA cross-linked to the protein of interest;
(viii) optionally releasing the antibody added in step (iv) by a washing step;
(ix) contacting the biotinylated DNA from step (vii) or, when present, from step (viii), with beads carrying the modified streptavidin according to the first aspect, thereby capturing the biotinylated DNA from step (vii) or, when present, from step (viii) and proteins cross-linked to the biotinylated DNA;
(x) optionally washing the beads with a wash buffer;
(xi) optionally adding a solution comprising a protease to the beads, thereby generating peptide fragments of proteins cross-linked to the biotinylated DNA;
(xii) optionally recovering the peptide fragments generated in step (xi); and
(xiii) optionally subjecting the peptide fragments recovered in step (xii) to mass spectroscopic analysis.

In a fifteenth aspect the present invention relates to a method for capturing chromatin-associated proteins, comprising the steps:
(i) providing cells the chromatin of which is to be investigated;
(ii) adding formaldehyde to the cells to crosslink chromatin;
(iii) shearing the chromatin sample, thereby generating a sheared chromatin sample;
(iv) adding biotinylated nucleotides and a DNA polymerase to the chromatin sample of step (iii), thereby biotinylating DNA within the sheared chromatin sample;
(v) contacting the biotinylated DNA from step (iv) with beads carrying the modified streptavidin according to the first aspect, thereby capturing the biotinylated DNA from step (iv) and proteins cross-linked to the biotinylated DNA;
(vi) optionally washing the beads with a wash buffer;
(vii) optionally adding a solution comprising a protease to the beads, thereby generating peptide fragments of proteins cross-linked to the biotinylated DNA;
(viii) optionally recovering the peptide fragments generated in step (vii); and
(ix) optionally subjecting the peptide fragments recovered in step (viii) to mass spectroscopic analysis.

This summary of the invention does not necessarily describe all features of the present invention. Other embodiments will become apparent from a review of the ensuing detailed description.

### BRIEF DESCRIPTION OF THE FIGURES

FIG. 1. Chromatograms of peptides generated by LysC and trypsin digestion from streptravidin-coated beads before and after blocking lysines and arginines.

A. Tryptic digestion of untreated streptavidin beads generates several peptides, the most abundant of which are indicated by the shown sequences.

B. After blocking of lysines via reductive methylation, streptavidin has become refractory to digestion by LysC evidenced by the absence of streptavidin-derived peptides.

C. After blocking of arginines and lysines (by cyclohexadione and reductive methylation, respectively), streptavidin has become refractory to digestion by trypsin.

FIG. 2. Capture and identification of the PRC2-complex bound to biotinylated DNA from a chromatin sample via LysC-resistant streptavidin beads.

A. Schematic representation of the PRC2-complex bound to biotinylated DNA and indication of method steps carried out for generation of peptide fragments from the proteins in the PRC2 complex.

Symbols: Triangle: Suz12; open ovals: proteins in the PRC2 complex; grey ovals: other transiently associated proteins; black line: DNA; solid black circle: biotin on DNA; inverted

Y: Suz12 antibody; inverted C: streptavidin (either unmodified or after blocking of Lysines (and optionally also Arginines).

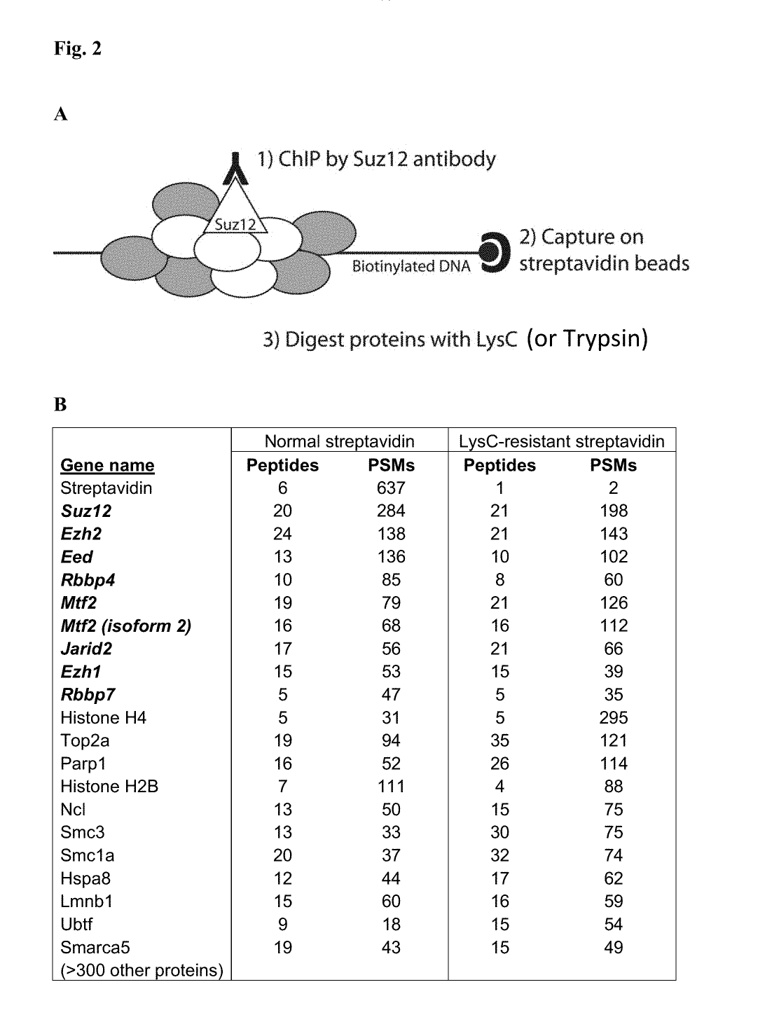

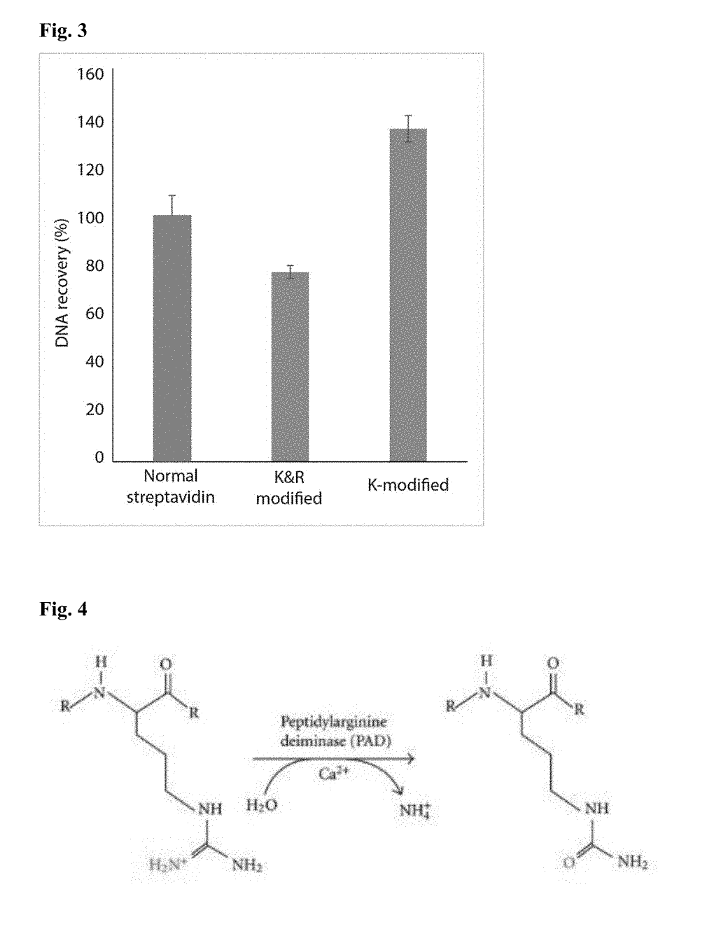

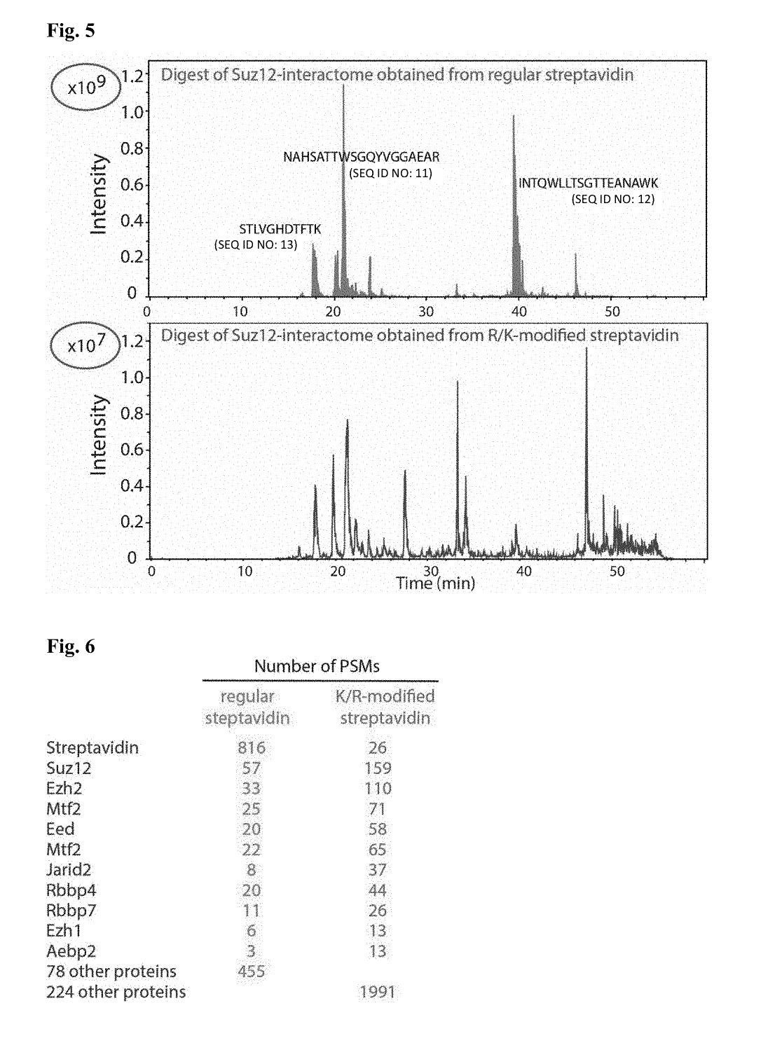

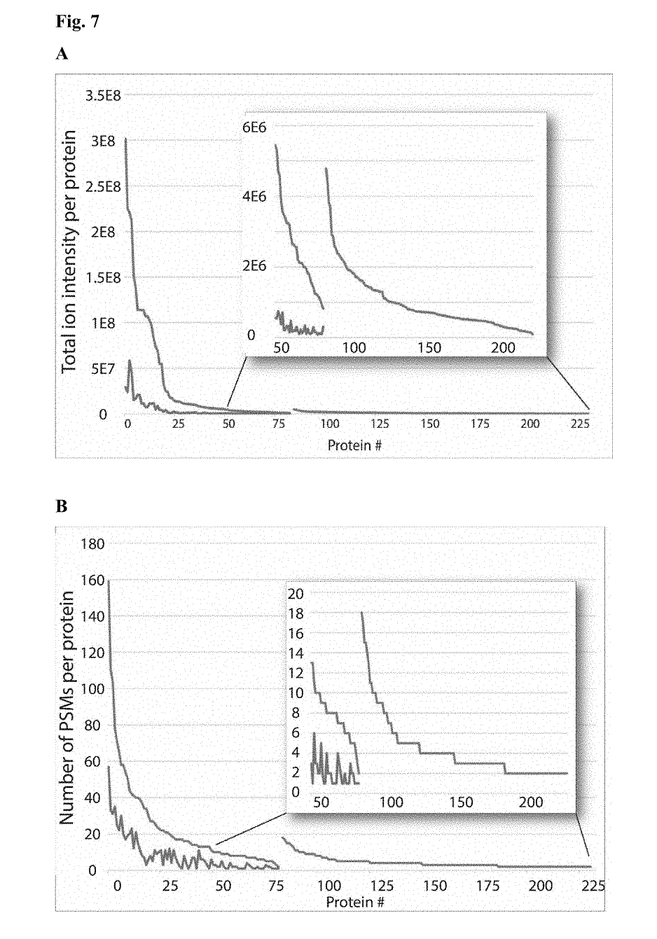

B. Proteins identified by mass spectrometry after carrying out the procedure described in FIG. 2A. Identified proteins are indicated by gene name, those known to belong to the PRC2 complex are indicated in bold italics. Peptides: number of peptides identified per protein. PSM: peptide-spectrum matches, indicating the number of times these peptides were identified per protein.

FIG. 3. Binding capacity of different types of streptavidin. The binding capacity of beads coated with different types of streptavidin was assessed by determining the recovery of biotinylated DNA. Left column: normal streptavidin; middle column: streptavidin with modified lysine and arginine residues; right column: streptavidin with modified lysine residues.

FIG. 4. Reaction scheme for the conversion of arginine residues to citrulline residues by peptidyl arginine deiminase (PAD).

FIG. 5. Capture and identification of the PRC2-complex bound to biotinylated DNA from a chromatin sample via trypsin-resistant streptavidin beads.

LC-MS chromatograms of peptides obtained after capture, elution and trypsin-digestion of the DNA-bound PRC2 complex.

Upper diagram: LC-MS chromatogram of peptides obtained when using regular streptavidin beads. The three most abundant sequences (all derived from streptavidin) are indicated by the shown sequences.

Lower diagram: LC-MS chromatogram of peptides obtained when using streptavidin beads with blocked Lysines and Arginines (blocked by reductive methylation and cyclohexadione, respectively).

FIG. 6. Capture and identification of the PRC2-complex bound to biotinylated DNA from a chromatin sample via trypsin-resistant streptavidin beads.

The table shows the number of peptide-spectrum matches (PSMs) in an analysis of the PRC2 complex enriched on regular streptavidin beads (left column, corresponding to upper diagram of FIG. 5) and on Lysine- and Arginine-modified streptavidin (right column, corresponding to lower diagram of FIG. 5).

FIG. 7. Use of modified streptavidin enables the detection of low-abundant proteins after affinity capture.

Ion intensity (panel A) and number of peptide-spectrum matches (panel B) of proteins identified after enrichment on regular streptavidin beads (red trace) and on Lysine- and Arginine-modified streptavidin beads (blue trace). The overall gain in sensitivity afforded by K&R-modified streptavidin (see FIG. 6) is the result of the consistent higher ion intensity for all proteins (panel A), and a larger number of PSMs for each of them (panel B).

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps.

The present invention relates to several methods that are defined by one or more method steps numbered by Roman numerals. The numbering of the methods steps does not necessarily imply that the individual steps have to be carried out in the order specified by the numbers. A person having ordinary skill in the art will know whether the order of steps may be changed or not, while still achieving the aim intended by the particular method.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

Streptavidin is a non-glycosylated bacterial protein produced by Streptomyces avidinii. The full-length sequence of streptavidin consists of 183 amino acids (shown as SEQ ID NO: 1 in the attached sequence listing) and contains a 24 amino acid signal peptide that is cleaved upon maturation to yield the mature form of streptavidin, which comprises 159 amino acids (shown as SEQ ID NO: 2 in the attached sequence listing). Streptavidin isolated from culture media of S. avidinii often have a truncated N-terminus and a truncated C-terminus due to postsecretory proteolytic digestion (cf. E.A. Bayer et al., Biochem J. (1989) 259:369-376; T. Sano et al., J. Biol. Chem. (1995) 270(47):28204-28209).

As used herein, the term “wild-type streptavidin” refers to the mature form of streptavidin with the amino acid sequence shown in SEQ ID NO: 2 of the sequence listing.

As used herein, the term “modified streptavidin” refers to a streptavidin molecule that is based on the naturally occurring streptavidin according to SEQ ID NO: 2 (or a naturally occurring truncated form of the amino acid sequence according to SEQ ID NO: 2) but contains modifications that may have been introduced into the streptavidin molecule, for example, by chemical modification of a naturally occurring streptavidin molecule, or by chemical synthesis of a streptavidin molecule containing such modifications, or by genetically modifying the streptavidin gene and expressing the modified gene in an appropriate expression host resulting in a modified amino acid sequence.

As used herein, the expression “chemical modification” does not only include modifications known to a person skilled in the art of organic chemistry but additionally includes biochemical modifications, such as modifications effected by enzymatic reactions. For example, arginine residues can be converted to citrulline residues by the enzymatic activity of peptidyl arginine deiminase (PAD) (see FIG. 4).

For the purpose of the present invention, a modified streptavidin is considered to be “resistant to cleavage by the protease . . . ” when the amount of cleavage products obtained upon incubation with the protease in question is only 25% or less of the amount of cleavage products that are obtained from a control proteolytic cleavage of the corresponding wild-type streptavidin incubated under identical conditions (same temperature, same buffer conditions, same amount of protease, same amount of wild-type streptavidin and modified streptavidin, etc.). If not specified otherwise, “amount” means molar amount in this context. However, the modified streptavidins of the present invention have almost the same molecular weight as the parent naturally occurring streptavidin molecule. Accordingly, there is little to no difference in the above definition regardless whether the amount is measured as molar amount (measured in mol, mmol, nmol etc.) or as mass amount (measured in g, mg or μg etc.). It is preferred that the amount of cleavage products obtained upon incubation with the protease in question is only 20% or less (preferably 15% or less; more preferably 10% or less, even more preferably 5% or less, still more preferably 2% or less, and most preferably 1% or less) of the amount of cleavage products that are obtained from a control proteolytic cleavage of the corresponding wild-type streptavidin incubated under identical conditions. In a narrower sense, the term “resistant to cleavage by the protease . . . ” means that the modified streptavidin is not cleaved at all by the protease in question.

As used herein, a first compound (e.g. streptavidin or an antibody) is considered to “bind” to a second compound (e.g. biotin or an antigen), if it has a dissociation constant K_{D} to said second compound of 1 μM or less, preferably 900 nM or less, preferably 800 nM or less, preferably 700 nM or less, preferably 600 nM or less, preferably 500 nM or less, preferably 400 nM or less, preferably 300 nM or less, preferably 200 nM or less, more preferably 100 nM or less, more preferably 90 nM or less, more preferably 80 nM or less, more preferably 70 nM or less, more preferably 60 nM or less, more preferably 50 nM or less, more preferably 40 nM or less, more preferably 30 nM or less, more preferably 20 nM or less, more preferably 10 nM or less, even more preferably 5 nM or less, even more preferably 4 nM or less, even more preferably 3 nM or less, even more preferably 2 nM or less, and even more preferably 1 nM or less.

The term “binding” according to the invention preferably relates to a specific binding. “Specific binding” means that a binding moiety (e.g. streptavidin or an antibody) binds stronger to a target for which it is specific (e.g. biotin or an antigen) as compared to the binding to another target. A binding moiety binds stronger to a first target compared to a second target if it binds to the first target with a dissociation constant (K_{D}) which is lower than the dissociation constant for the second target. Preferably the dissociation constant (K_{D}) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (K_{D}) for the target to which the binding moiety does not bind specifically.

As used herein, the term “K_{D}” (usually measured in “mol/L”, sometimes abbreviated as “M”) is intended to refer to the dissociation equilibrium constant of the particular interaction between a first molecule (e.g. streptavidin) and a second molecule (e.g. biotin). Methods for determining binding affinities, i.e. for determining the dissociation constant K_{D}, are known to a person of ordinary skill in the art and can be selected for instance from the following methods known in the art: Surface Plasmon Resonance (SPR) based technology, Bio-layer interferometry (BLI), enzyme-linked immunosorbent assay (ELISA), flow cytometry, fluorescence spectroscopy techniques, isothermal titration calorimetry (ITC), analytical ultracentrifugation, radioimmunoassay (RIA or IRMA) and enhanced chemiluminescence (ECL). Binding affinity between biotin and modified streptavidins can also be determined by contacting the modified streptavidin with a biotinylated DNA probe. The amount of bound DNA probe can be determined using quantitative PCR as read-out. Typically, the dissociation constant K_{D} is determined at 20° C., 25° C. or 30° C. If not specifically indicated otherwise, the K_{D} values recited herein are determined at 25° C. by ELISA.

As used herein, the term “binding capacity” refers to the maximum amount (mass amount or molar amount) of a target molecule that can be bound to a support material. Typically, the “binding capacity” is calculated in comparison to the number, the area or the amount of the support material. For example, the binding capacity of a support material may be expressed as μg/bead or μg/cm² or μg/g of support material.

Unless the context dictates otherwise, the terms “peptide”, “polypeptide” and “protein” are used interchangeably herein and refer to a linear molecular chain of at least two amino acids linked by peptide bonds.

In certain embodiments of the present invention, the term “peptide” refers to a linear molecular chain of between two and 100 amino acids linked by peptide bonds. In these embodiments, the terms “protein” and “polypeptide” refer to any linear molecular chain of more than 100 amino acids linked by peptide bonds. The terms “polypeptide” and “protein” are always used interchangeably herein. The term “polypeptide” is also intended to refer to the products of post-translational modifications of the polypeptide, including without limitation glycosylation, acetylation, phosphorylation, amidation, proteolytic cleavage, modification by non-naturally occurring amino acids and similar modifications which are well-known in the art.

As used herein, the term “protein interaction partner” refers to a protein that interacts with a molecule of interest. Thus, the protein and the molecule of interest can be termed interaction partners. In this context, “interaction” typically means that the protein binds to the molecule of interest and in particular shows specific binding to the molecule of interest; wherein the terms “binding” and “specific binding” have the meaning as defined above. As used herein, the term “protein interaction partner” does not only refer to proteins consisting of a single polypeptide chain but also relates to protein complexes comprising two or more polypeptide chains (also termed “subunits”). For some embodiments described herein, it may favorable or necessary to covalently link the subunits of a protein complex to each other. For example, the subunits of a protein complex may be cross-linked with formaldehyde or glutaraldehyde. In another example, the molecule of interest can be a nucleic acid molecule (e.g. DNA or RNA) and the nucleic acid may be cross-linked to a protein interaction partner, such as a DNA-binding protein (wherein said DNA-binding protein may consists of a single peptide or may comprise two or more subunits). The term “PSM” is the abbreviation for peptide spectrum matches, i.e. the assignment of a fragmentation pattern observed by MS to a peptide identity.

As used herein, the term “small molecule” refers to an organic or inorganic compound of a molar mass lower than 1.000 g/mol, preferably lower than 500 g/mol. “Small molecules” within the meaning of the present invention are non-peptidic (i.e. no peptide bonds) and non-nucleic acid compounds.

As used herein, the term “oligonucleotide” refers to a nucleic acid molecule comprising between 2 and 100 nucleotides covalently linked to each other.

As used herein, the term “polynucleotide” refers to a nucleic acid molecule comprising more than 100 nucleotides covalently linked to each other. Nucleic acid molecules (i.e. oligonucleotides or polynucleotides) usable in the present invention will generally contain phosphodiester bonds, although in some cases nucleic acid analogs are included that may have alternate backbones, comprising, for example, phosphoramide, phosphorothioate, phosphorodithioate, O-methylphosphoroamidite linkages, and peptide nucleic acid backbones and linkages. Other analog nucleic acids include those with positive backbones, non-ionic backbones and non-ribose backbones. Nucleic acids containing one or more carbocyclic sugars are also included within the definition of nucleic acids. These modifications of the ribose-phosphate backbone may be done to facilitate the addition of labels, or to increase the stability and half-life of such molecules in physiological environments. Nucleic acids usable in the context of the present invention can consist of DNA, RNA, peptide nucleic acid (PNA), phosphorothioate DNA (PS-DNA), 2′-O-methyl RNA (OMe-RNA), 2′-O-methoxy-ethyl RNA (MOE-RNA), N3′-P5′ phosphoroamidate (NP), 2′-fluoro-arabino nucleic acid (FANA), locked nucleic acid (LNA), morpholinophosphoroamidate (MF), cyclohexene nucleic acid (CeNA), or tricycle-DNA (tcDNA) or of mixtures of any of these naturally occurring nucleic acids and nucleic acid analogs. As will be appreciated by those skilled in the art, all of these nucleic acid analogs may find use in the present invention. In addition, mixtures of naturally occurring nucleic acids, such as DNA and RNA, and analogs can be prepared. Alternatively, mixtures of different nucleic acid analogs, and mixtures of naturally occurring nucleic acids and analogs can be made.

### Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

In a first aspect the present invention is directed to a modified streptavidin that
(i) is resistant to cleavage by at least one endopeptidase, wherein said at least one endopeptidase is specific for a basic amino acid; and
(ii) exhibits a dissociation constant K_{D} to biotin of 10⁻¹⁰ M or less.

In some embodiments of the first aspect, said at least one endopeptidase is selected from the group consisting of LysC, LysN, ArgC, and trypsin. In particular embodiments of the first aspect, the modified streptavidin is resistant to cleavage by both LysC and trypsin.

In one embodiment of the first aspect, the modified streptavidin exhibits a dissociation constant K_{D} to biotin in the range between 10⁻¹⁵ M and 10⁻¹⁰ M; for example in the range between 10⁻¹⁴ M and 10⁻¹¹ M, or in the range between 10⁻¹³ M and 10⁻¹² M.

In one embodiment of the first aspect, said at least one endopeptidase is selected from the group consisting of LysC, LysN, and trypsin, and one or more lysine residues carry at least one chemical modification selected from the group consisting of:
(i) a chemical modification that neutralizes the positive charge of the side chain in Lysines; and
(ii) a chemical modification that replaces a hydrogen of the ε amino group in Lysines thus converting the primary amine to a secondary or tertiary amine.

Such chemical modifications of lysine residues—and in particular chemical modifications of the ε-amino group of lysine residues—are described in“The Protein protocols handbook” (2009), 3rd edition. Walker J.M. (Ed.), Humana press.

For example, the amino side chain can be acylated (using e.g., acetic anhydride) or alkylated by trinitrobenzenesulfonic acid (TNBS); these reactions alter both the size and the charge of the amino group. Other modifications, using anhydrides of dicarboxylic acids (e.g., succinic anhydride), replace the positively charged amino group with a negatively charged carboxyl group. Amidinations and reductive alkylations offer an opportunity to modify the structure of the ε-amino group of lysines, while maintaining the positive charge.

Thus, in further embodiments of the first aspect, said chemical modification is produced by a chemical reaction selected from the group consisting of:
(i) acylation of lysine residues producing acyl-lysine; preferably acetylation of lysine residues producing acetyl-lysine (e.g. by using acetic anhydride);
(ii) reductive alkylation of lysine residues producing dialkyl-lysine; preferably reductive methylation of lysine residues producing dimethyl-lysine;
(iii) reaction of lysine residues with propionic anhydride producing propionyl lysine;
(iv) reaction of lysine residues with succinic anhydride producing lysine dicarboxylic anhydride;
(v) alkylation of lysine residues producing alkyl-lysine;
(vi) amidination of lysine residues producing the acetimidine derivative of lysine.

In one embodiment of the first aspect, said at least one endopeptidase is selected from the group consisting of LysC, LysN, and trypsin, and the modified streptavidin is a mutein of the wild-type streptavidin amino acid sequence according to SEQ ID NO: 2, wherein said mutein is characterized by amino acid exchanges at least in positions K121 and K132 of SEQ ID NO: 2 (corresponding to K145 and K156 of SEQ ID NO: 1, respectively), and wherein said mutein optionally comprises between 1 and 10 (e.g. 1, 2, 3, 4, 5, 6, 7, 8, 9, or 10) internal amino acid deletions, optionally comprises between 1 and 10 (e.g. 1, 2, 3, 4, 5, 6, 7, 8, 9, or 10) amino acid insertions, optionally comprises between 1 and 10 (e.g. 1, 2, 3, 4, 5, 6, 7, 8, 9, or 10) amino acid exchanges, optionally comprises between 1 and 13 (e.g. 1, 2, 3, 4, 5, 6, 7, 8, 9, 10, 11, 12, or 13) N-terminal deletions, and/or optionally comprises between 1 and 20 (e.g. 1, 2, 3, 4, 5, 6, 7, 8, 9, 10, 11, 12, 13, 14, 15, 16, 17, 18, 19, or 20) C-terminal deletions.

In preferred embodiments of the first aspect, the modified streptavidin is a mutein of the wild-type streptavidin amino acid sequence according to SEQ ID NO: 2, wherein said mutein is characterized by amino acid exchanges at least in positions K121 and K132 of SEQ ID NO: 2 (corresponding to K145 and K156 of SEQ ID NO: 1, respectively), and wherein said mutein optionally comprises between 1 and 13 (e.g. 1, 2, 3, 4, 5, 6, 7, 8, 9, 10, 11, 12, or 13) N-terminal deletions and/or optionally comprises between 1 and 20 (e.g. 1, 2, 3, 4, 5, 6, 7, 8, 9, 10, 11, 12, 13, 14, 15, 16, 17, 18, 19, or 20) C-terminal deletions. Thus, in these embodiments, the modified streptavidin does not contain internal amino acid deletions or amino acid insertions or amino acid exchanges other than the amino acid exchanges in positions K121 and K132.

In further embodiments of the first aspect, K121 and K132 have been replaced, independently from each other, by another amino acid, wherein said another amino acid is neither lysine nor arginine. The amino acid replacing K121 or K132 can be any amino acid, be it a naturally occurring amino acid or an artificial amino acid, provided that said replacing amino acid is neither lysine nor arginine. Preferably, the replacing amino acid does not carry a positive charge and does not carry hydrogen residues that are capable of making hydrogen bonds in the active site of an endopeptidase described herein.

In further embodiments of the first aspect, the amino acids replacing K121 or K132 are, independently from each other, selected from the group consisting of alanine, asparagine, aspartic acid, cysteine, glutamic acid, glutamine, glycine, histidine, isoleucine, leucine, methionine, phenylalanine, proline, serine, threonine, tryptophan, tyrosine, valine, hydroxy-proline, citrulline, acetyl-ornithine, acetamido-methyl-cysteine, O-acetamido-methyl-homo-serine, S-acetamido-methyl-homo-cysteine, acetyl-lysine, propionyl-lysine, hydroxyl-acetyl-lysine, monofluoroacetyl-lysine, difluoroacetyl-lysine, trifluoroacetyl-lysine, crotonyl-lysine, and dimethyl-lysine.

In some embodiments of the first aspect, the at least one endopeptidase is selected from the group consisting of ArgC and trypsin, and one or more arginine residues carry at least one chemical modification selected from the group consisting of: (i) a chemical modification that neutralizes the positive charge of the guanidinium group; and (ii) a chemical modification that replaces one or more hydrogens of the guanidinium group. In some embodiments of the first aspect, said chemical modification is produced by a chemical reaction selected from the group consisting of: (i) reaction of arginine residues with dicarbonyl compounds producing a modified arginine residue; (ii) carbamylation of arginine residues producing carbamylated arginines; and (iii) de-imination of arginine residues producing citrulline residues. Dicarbonyl compounds particularly suitable for reaction (i) are α-dicarbonyl compounds and include dialdehydes, ketoaldehydes, and diketones. Suitable α-dicarbonyl compounds include, but are not limited to, biacetyl, pyruvic acid, glyoxal, methylglyoxal, deoxyosones, 3-deoxyosones, malondialdehyde, 2-oxopropanal, phenylglyoxal, 2,3-butanedione, and 1,2-cyclohexanedione. The chemical reaction between arginine residues and dicarbonyl compounds is described in WO 2004/046314 A2. A reagent suitable for carrying out the carbamylation reaction (ii) is isocyanic acid. The de-imination of arginine residues according to reaction (iii) can be carried out biochemically by enzymatic reaction with peptidyl arginine deiminase (PAD) (see FIG. 4).

In one embodiment of the first aspect, the at least one endopeptidase is selected from the group consisting of ArgC and trypsin, and the modified streptavidin is a mutein of the wild-type streptavidin amino acid sequence according to SEQ ID NO: 2, wherein said mutein is characterized by one or more amino acid exchanges in positions R59, R84, and R103 of SEQ ID NO: 2 (corresponding to R83, R108, or R127 of SEQ ID NO: 1), and wherein said mutein optionally comprises between 1 and 10 (e.g. 1, 2, 3, 4, 5, 6, 7, 8, 9, or 10) internal amino acid deletions, optionally comprises between 1 and 10 (e.g. 1, 2, 3, 4, 5, 6, 7, 8, 9, or 10) amino acid insertions, optionally comprises between 1 and 10 (e.g. 1, 2, 3, 4, 5, 6, 7, 8, 9, or 10) amino acid exchanges, optionally comprises between 1 and 13 (e.g. 1, 2, 3, 4, 5, 6, 7, 8, 9, 10, 11, 12, or 13) N-terminal deletions, and/or optionally comprises between 1 and 20(e.g. 1, 2, 3, 4, 5, 6, 7, 8, 9, 10, 11, 12, 13, 14, 15, 16, 17, 18, 19, or 20) C-terminal deletions.

The amino acid exchanges in positions R59, R84, and R103 of SEQ ID NO: 2 (corresponding to R83, R108, or R127 of SEQ ID NO: 1) may be present in addition to the amino acid exchanges in positionsK121 and K132 of SEQ ID NO: 2 (corresponding to K145 and K156 of SEQ ID NO: 1, respectively).

In preferred embodiments of the first aspect, the modified streptavidin is a mutein of the wild-type streptavidin amino acid sequence according to SEQ ID NO: 2, wherein said mutein is characterized by one or more amino acid exchanges in positions R59, R84, and R103 of SEQ ID NO: 2 (corresponding to R83, R108, or R127 of SEQ ID NO: 1), and wherein said mutein optionally comprises between 1 and 13 (e.g. 1, 2, 3, 4, 5, 6, 7, 8, 9, 10, 11, 12, or 13) N-terminal deletions, and/or optionally comprises between 1 and 20 (e.g. 1, 2, 3, 4, 5, 6, 7, 8, 9, 10, 11, 12, 13, 14, 15, 16, 17, 18, 19, or 20) C-terminal deletions. Thus, in these embodiments, the modified streptavidin does not contain internal amino acid deletions or amino acid insertions or amino acid exchanges other than the amino acid exchanges in positions R59, R84, R103, K121, or K132.

In preferred embodiments of the first aspect, R59, R84, and/or R103 have been replaced, independently from each other, by another amino acid, wherein said another amino acid is neither lysine nor arginine. The amino acid replacing R59, R84, or R103 can be any amino acid, be it a naturally occurring amino acid or an artificial amino acid, provided that said replacing amino acid is neither lysine nor arginine. Preferably, the replacing amino acid does not carry a positive charge and does not carry hydrogen residues that are capable of making hydrogen bonds in the active site of an endopeptidase described herein.

In further embodiments of the first aspect, the amino acids replacing R59, R84, or R103 are, independently from each other, selected from the group consisting of alanine, asparagine, aspartic acid, cysteine, glutamic acid, glutamine, glycine, histidine, isoleucine, leucine, methionine, phenylalanine, proline, serine, threonine, tryptophan, tyrosine, valine, hydroxy-proline, citrulline, acetyl-ornithine, acetamido-methyl-cysteine, O-acetamido-methyl-homo-serine, S-acetamido-methyl-homo-cysteine, acetyl-lysine, propionyl-lysine, hydroxyl-acetyl-lysine, monofluoroacetyl-lysine, difluoroacetyl-lysine, trifluoroacetyl-lysine, crotonyl-lysine, and dimethyl-lysine.

In a second aspect the present invention is directed to a nucleic acid molecule comprising a nucleotide sequence which encodes the modified streptavidin according to the first aspect. As explained above, the modified streptavidin according to the first aspect encompasses streptavidin molecules obtainable by chemical modification of natural or artificial streptavidin molecules, streptavidin molecules obtainable by chemical synthesis, and streptavidin molecules obtainable by genetic engineering. As will be understood by a person skilled in the art, the second aspect only pertains to a nucleic acid molecule comprising a nucleotide sequence which encodes a modified streptavidin molecule of the first aspect that is obtainable by genetic engineering. Thus, the nucleic acid molecule preferably comprises a nucleotide sequence encoding a mutein of the wild-type streptavidin amino acid sequence according to SEQ ID NO: 2 as described above.

In a third aspect the present invention is directed to a vector comprising the nucleic acid molecule according to the second aspect. In preferred embodiments, the vector is an expression vector.

In a fourth aspect the present invention is directed to a cell, preferably a host cell, more preferably an isolated host cell, comprising the nucleic acid molecule of the second aspect or the vector (or expression vector) of the third aspect.

A host cell is a cell that has been transformed, or is capable of being transformed, with a nucleic acid sequence and thereby expresses a gene of interest. Suitable host cells include prokaryotes or eukaryotes. Various mammalian or insect cell culture systems can also be employed to express recombinant proteins.

In a fifth aspect the present invention is directed to a solid support comprising the modified streptavidin of the first aspect.

In a preferred embodiment of the fifth aspect, the solid support is selected from the group consisting of beads, tubes, chips, resins, plates, wells, films, sticks, magnetic beads, porous membranes and combinations thereof.

In a sixth aspect the present invention is directed to a kit comprising the modified streptavidin of the first aspect or the solid support of the fifth aspect and further comprising at least one protease selected from the group consisting of LysC, LysN, ArgC, and trypsin.

Preferably, the kit comprises LysC and optionally comprises at least one protease selected from the group consisting of LysN, ArgC, and trypsin.

Alternatively, the kit comprises trypsin and optionally comprises at least one protease selected from the group consisting of LysC, LysN, and ArgC.

In a preferred embodiment of the sixth aspect, the kit further comprises one or more components selected from the group consisting of a buffer solution appropriate for the protease present in the kit (i.e. a buffer appropriate for LysC, a buffer appropriate for LysN, a buffer appropriate for ArgC, and/or a buffer appropriate for trypsin), a protein standard (preferably a biotinylated protein standard), reagents for sample clean-up after digestion, and instructions for use.

In a seventh aspect the present invention is directed to a use of the modified streptavidin of the first aspect or the solid support of the fifth aspect or the kit of the sixth aspect for capture or immobilization of at least one biotinylated molecule.

In preferred embodiments of the seventh aspect, the at least one biotinylated molecule is selected from the group consisting of proteins, peptides, oligonucleotides (e.g. aptamers), polynucleotides (e.g. DNA, RNA, or PNA), lipids, (poly)saccharides, carbohydrates, metabolites, drugs, small molecules, natural and synthetic molecules.

In an eighth aspect the present invention is directed to a use of the modified streptavidin of the first aspect or the solid support of the fifth aspect or the kit of the sixth aspect for protein purification.

In a ninth aspect the present invention is directed to a use of the modified streptavidin of the first aspect or the solid support of the fifth aspect or the kit of the sixth aspect in mass spectrometry.

In preferred embodiments of the ninth aspect, said use is for reducing background in mass spectrometry.

In a tenth aspect the present invention is directed to a method for reducing background in mass spectrometry, comprising the steps:
(i) providing beads carrying the modified streptavidin according to the first aspect;
(ii) contacting a sample comprising a biotinylated protein with the beads of step (i), thereby binding the biotinylated protein to the modified streptavidin;
(iii) optionally washing the beads with a wash buffer;
(iv) adding a solution comprising a protease to the beads, thereby generating peptide fragments of the biotinylated protein;
(v) recovering the peptide fragments generated in step (iv); and
(vi) optionally subjecting the peptide fragments recovered in step (v) to mass spectroscopic analysis.

In some embodiments of the tenth aspect, step (iii) is carried out to remove proteins and other undesired material, in particular to remove non-biotinylated proteins.

In some embodiments of the tenth aspect, step (iv) is carried out at a temperature and for a time-period sufficient to achieve proteolytic digestion of the biotinylated protein.

In an eleventh aspect the present invention is directed to a method for reducing background in mass spectrometry, comprising the steps:
(i) providing beads carrying the modified streptavidin according to the first aspect;
(ii) contacting a sample comprising a biotinylated protein with the beads of step (i), thereby binding the biotinylated protein to the modified streptavidin;
(iii) optionally washing the beads with a wash buffer;
(iv) eluting the biotinylated protein;
(v) adding a solution comprising a protease to the biotinylated protein eluted in step (iv), thereby generating peptide fragments of the biotinylated protein;
(vi) recovering the peptide fragments generated in step (v); and
(vii) optionally subjecting the peptide fragments recovered in step (vi) to mass spectroscopic analysis.

In some embodiments of the eleventh aspect, step (iii) is carried out to remove proteins and other undesired material, in particular to remove non-biotinylated proteins.

In some embodiments of the tenth aspect, step (v) is carried out at a temperature and for a time-period sufficient to achieve proteolytic digestion of the biotinylated protein.

In a twelfth aspect the present invention is directed to a method for capturing a protein interaction partner of a molecule, comprising the steps:
(i) providing beads carrying the modified streptavidin according to the first aspect;
(ii) contacting a biotinylated molecule with the beads of step (i), thereby loading the biotinylated molecule onto the beads;
(iii) contacting a sample with the beads loaded with the biotinylated molecule obtained in step (ii), wherein said sample comprises at least one protein interaction partner for the biotinylated molecule;
(iv) optionally washing the beads with a wash buffer;
(v) adding a solution comprising a protease to the beads, thereby generating peptide fragments of the at least one protein interaction partner;
(vi) recovering the peptide fragments generated in step (v); and
(vii) optionally subjecting the peptide fragments recovered in step (v) to mass spectroscopic analysis.

In some embodiments of the twelfth aspect, step (iv) is carried out to remove proteins and other undesired material, in particular to remove proteins that do not bind to the biotinylated molecule.

In some embodiments of the twelfth aspect, step (v) is carried out at a temperature and for a time-period sufficient to achieve proteolytic digestion of the protein interaction partner.

In a thirteenth aspect the present invention is directed to a method for capturing a protein interaction partner of a molecule, comprising the steps:
(i) providing beads carrying the modified streptavidin according to the first aspect;
(ii) contacting a biotinylated molecule with the beads of step (i), thereby loading the biotinylated molecule onto the beads;
(iii) contacting a sample with the beads loaded with the biotinylated molecule obtained in step (ii), wherein said sample comprises at least one protein interaction partner for the biotinylated molecule;
(iv) optionally washing the beads with a wash buffer;
(v) eluting the at least one protein interaction partner;
(vi) adding a solution comprising a protease to the protein interaction partner eluted in step (v), thereby generating peptide fragments of the at least one protein interaction partner;
(vii) recovering the peptide fragments generated in step (vi); and
(viii) optionally subjecting the peptide fragments recovered in step (vii) to mass spectroscopic analysis.

In some embodiments of the thirteenth aspect, step (iv) is carried out to remove proteins and other undesired material, in particular to remove proteins that do not bind to the biotinylated molecule.

In some embodiments of the thirteenth aspect, step (vi) is carried out at a temperature and for a time-period sufficient to achieve proteolytic digestion of the protein interaction partner.

In some embodiments of the twelfth and thirteenth aspect, the biotinylated molecule is selected from the group consisting of proteins, peptides, oligonucleotides (e.g. aptamers), polynucleotides (e.g. DNA, RNA, or PNA), lipids, (poly)saccharides, carbohydrates, metabolites, drugs and small molecules, natural and synthetic molecules. In the context of the present invention, the term “molecule” may also refer to complexes of different molecules or different types of molecules that have been connected to each other, e.g. via cross-linking. For example, as used herein the term “molecule” may also refer to subunits from the same protein covalently linked to each other by cross-linking, to different proteins covalently linked to each other by cross-linking, or to proteins and polynucleotides covalently linked to each other by cross-linking.

Thus, in some embodiments of the twelfth and thirteenth aspect, the biotinylated molecule is a complex comprising biotinylated DNA as well as chromatin-associated proteins that are cross-linked to each other and/or to the biotinylated DNA. A procedure for the preparation of such a complex comprising biotinylated DNA and chromatin-associated proteins is described below in the fourteenth aspect and in Example 3.

In a fourteenth aspect the present invention is directed to a method for capturing chromatin-associated proteins, comprising the steps:
(i) providing cells the chromatin of which is to be investigated;
(ii) adding formaldehyde to the cells to crosslink chromatin;
(iii) shearing the chromatin sample, thereby generating a sheared chromatin sample;
(iv) adding an antibody that is specific for a chromatin-associated protein of interest to the cross-linked and sheared chromatin-sample of step (iii), thereby immuno-precipitating the protein of interest and molecules cross-linked to the protein of interest;
(v) contacting the immuno-precipitated protein from step (iv) with first beads coated with protein A or protein G, thereby immobilizing the immuno-precipitated protein on the beads;
(vi) optionally washing the beads with a wash buffer;
(vii) adding biotinylated nucleotides and a DNA polymerase to the immuno-precipitated protein of step (v) or, when present, of step (vi), thereby biotinylating DNA cross-linked to the protein of interest;
(viii) optionally releasing the antibody added in step (iv) by a washing step;
(ix) contacting the biotinylated DNA from step (vii) or, when present, from step (viii), with second beads carrying the modified streptavidin according to the first aspect, thereby capturing the biotinylated DNA from step (vii) or, when present, from step (viii) and proteins cross-linked to the biotinylated DNA;
(x) optionally washing the beads with a wash buffer;
(xi) optionally adding a solution comprising a protease to the beads, thereby generating peptide fragments of proteins cross-linked to the biotinylated DNA;
(xii) optionally recovering the peptide fragments generated in step (xi); and
(xiii) optionally subjecting the peptide fragments recovered in step (xii) to mass spectroscopic analysis.

The mass spectroscopic analysis of the peptide fragments allows the identification of the proteins from which the peptides were derived and thus allows the identification of chromatin-associated proteins interacting with the protein of interest.

In preferred embodiments of the fourteenth aspect, a centrifugation step is performed between steps (iii) and (iv) to collect the sheared chromatin.

In an alternative embodiment of the fourteenth aspect, the digestion with the protease does not take place on the second beads but only after elution of the biotinylated DNA (and the proteins cross-linked thereto) from the beads.

In a fifteenth aspect, the present invention is directed to a method for capturing chromatin-associated proteins, comprising the steps:
(i) providing cells the chromatin of which is to be investigated;
(ii) adding formaldehyde to the cells to crosslink chromatin;
(iii) shearing the chromatin sample, thereby generating a sheared chromatin sample;
(iv) adding biotinylated nucleotides and a DNA polymerase to the chromatin sample of step (iii), thereby biotinylating DNA within the sheared chromatin sample;
(v) contacting the biotinylated DNA from step (iv) with beads carrying the modified streptavidin according to the first aspect, thereby capturing the biotinylated DNA from step
(iv) and proteins cross-linked to the biotinylated DNA;
(vi) optionally washing the beads with a wash buffer;
(vii) optionally adding a solution comprising a protease to the beads, thereby generating peptide fragments of proteins cross-linked to the biotinylated DNA;
(viii) optionally recovering the peptide fragments generated in step (vii); and
(ix) optionally subjecting the peptide fragments recovered in step (viii) to mass spectroscopic analysis.

The mass spectroscopic analysis of the peptide fragments allows the identification of the proteins from which the peptides were derived and thus allows the identification of essentially all chromatin-associated proteins.

In preferred embodiments of the fifteenth aspect, a centrifugation step is performed between steps (iii) and (iv) to collect the sheared chromatin.

In an alternative embodiment of the fifteenth aspect, the digestion with the protease does not take place on the beads but only after elution of the biotinylated DNA (and the proteins cross-linked thereto) from the beads.

In preferred embodiments of the tenth, eleventh, twelfth, or thirteenth aspect, the sample is selected from the group consisting of blood, serum, plasma, urine, tissue, cell-culture supernatants, and cell lysates.

In preferred embodiments of the tenth, eleventh, twelfth, thirteenth, fourteenth, or fifteenth aspect, the protease is an endopeptidase selected from the group consisting of LysC, LysN, ArgC, and trypsin. It is preferred that the protease is LysC or trypsin.

In preferred embodiments of the tenth, eleventh, twelfth, thirteenth, fourteenth, or fifteenth aspect, the modified streptavidin is covalently bound to the beads.

In alternative embodiments of the tenth, eleventh, twelfth, thirteenth, fourteenth, or fifteenth aspect, the modified streptavidin is not present on beads but rather on a support material within a chromatography column.

### EXAMPLES

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees centigrade, and pressure is at or near atmospheric.

### Example 1

### Reductive Methylation of Lysine Residues in Streptavidin

The chemical reaction for the blocking of lysine residues is based on the reductive methylation method described by Boersema et al. with the modifications described below (Boersema P.J. et al. (2009) Nat. Protoc. 4(4):484-94).

The blocking reaction is carried out with the streptavidin already attached to beads. Subsequently, biotinylated proteins are bound to the blocked streptavidin and the samples are digested with the protease LysC, which cleaves the bound proteins at lysine residues without touching streptavidin.

### Reagents

4.5 ml of 50 mM sodium phosphate buffer pH 7.5

250 μl of 600 mM NaBH3CN

250 μl of 4% formaldehyde

Acetic acid (Merck, cat. no. 1.00063)

Acetonitrile (ACN) (Biosolve, cat. no. 75-05-8)

Ammonia solution (25% (vol/vol), Merck, cat. no. 1.05432)

Formaldehyde (CH2O) (37% (vol/vol), Sigma, cat. no. 252549)

Formic acid (Merck, cat. no. 1.00264)

Sodium cyanoborohydride (NaBH₃CN) (Fluka, cat. no. 71435)

Sodium cyanoborodeuteride (NaBD₃CN) (96% D, Isotec, cat. no. 190020)

Sodium dihydrogen phosphate (NaH2PO4) (Merck, cat. no. 1.06346)

Di-sodium hydrogen phosphate (Na2HPO4) (Merck, cat. no. 1.06580)

Triethylammoniumbicarbonate (TEAB)

### Protocol

1. reconstitute the sample in 100 μl of 100 mM TEAB

2. add 4 μl of 4% formaldehyde

3. mix briefly and spin

4. add 4 μl of 600 mM NaBH3CN

5. mix for 1 h at RT

6. quench reaction by adding 16 μl of 1% ammonia solution

7. mix briefly and spin

8. add 8 μl of formic acid to further quench and to acidify

9. dry down and analyse

### Results

The most critical lysines that have to be blocked are the ones indicated below by underlining (K121 and K132, corresponding to K145 and K156, respectively, of SEQ ID NO: 1):

(SEQ ID NO: 2)
DPSKDSKAQV SAAEAGITGT WYNQLGSTFI VTAGADGALT
GTYESAVGNA ESRYVLTGRY DSAPATDGSG TALGWTVAWK
NNYRNAHSAT TWSGQYVGGA EARINTQWLL TSGTTEANAW
KSTLVGHDTF TKVKPSAASI DAAKKAGVNN GNPLDAVQQ

Without wishing to be bound by a particular theory, the inventors believe that other lysine residues are not accessible by LysC, or at least cleavage is less efficient.

### Example 2

### Peptides Identified from Streptavidin before and after Chemical Blocking of Arginines and Lysines

Lysine and Arginine residues in streptavidin were blocked by subsequent reactions with reductive methylation and cyclohexadione, respectively. The blocking reactions were carried out with the streptavidin already attached to the beads. Subsequently, the streptavidin beads were subjected to tryptic digestion. The obtained peptide fragments were analysed by LC-MS.

TABLE 1
Tryptic peptides generated
from un-modified streptavidin

| | | number |
|---|---|---|
| mass | | of |
| (Da) | Sequence | spectra |
| 1157.65 | K.VKPSAASIDAAK.K | 1 |
| | (SEQ ID NO: 6) | |
| 1205.62 | K.STLVGHDTFTK.V | 198 |
| | (SEQ ID NO: 5) | |
| 1962.91 | R.NAHSATTWSGQYVGGAEAR.I | 88 |
| | (SEQ ID NO: 3) | |
| 2034.03 | R.INTQWLLTSGTTEANAWK.S | 108 |
| | (SEQ ID NO: 4) | |
| 2154.01 | R.YDSAPATDGSGTALGWTVAWK.N | 4 |
| | (SEQ ID NO: 7) | |
| 2510.16 | K.NNYRNAHSATTWSGQYVGGAEA | 8 |
| | R.I (SEQ ID NO: 8) | |
| 2843.4 | R.YVLTGRYDSAPATDGSGTALGWT | 3 |
| | VAWK.N (SEQ ID NO: 9) | |
| 3220.63 | R.INTQWLLTSGTTEANAWKSTLVG | 1 |
| | HDTFTK.V (SEQ ID NO: 10) | |
| | Total | 411 |

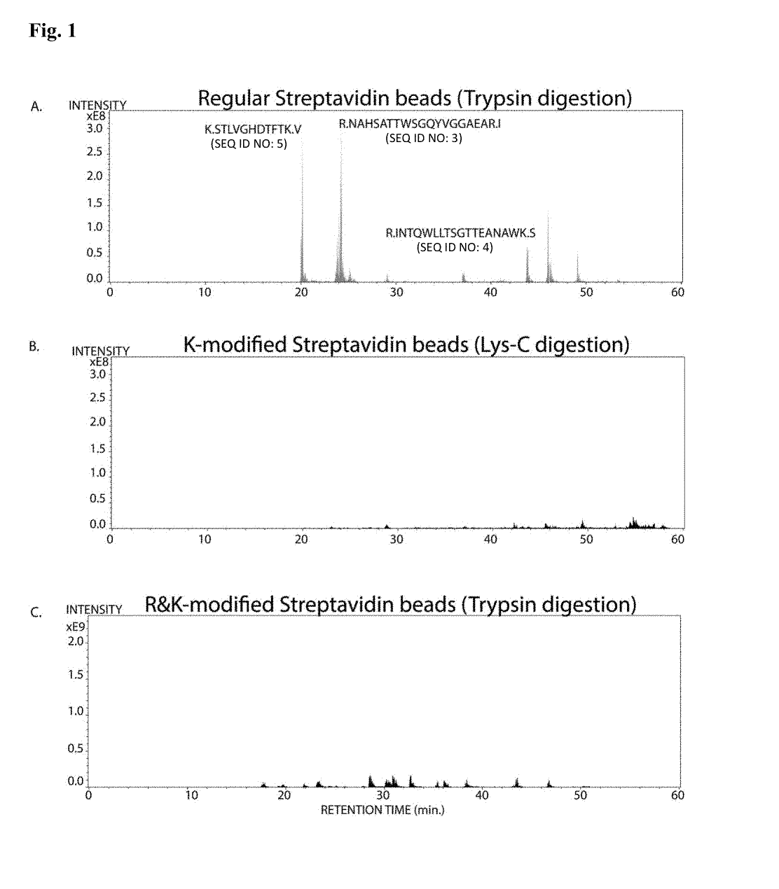

Tryptic digestion of unmodified streptavidin beads followed by analysis via LC-MS results in the identification of multiple peptides (see also FIG. 1A). In total, 411 spectra were recorded, most of them multiple times, indicating their high abundance (Table 1).

TABLE 2
Tryptic peptides generated from streptavidin
after chemical blocking of K's and R's

| | | number |
|---|---|---|
| mass | | of |
| (Da) | Sequence | spectra |
| 1962.91 | R.NAHSATTWSGQYVGGAEAR.I | 2 |
| | (SEQ ID NO: 3) | |
| 2154.01 | R.YDSAPATDGSGTALGWTVAWK.N | 1 |
| | (SEQ ID NO: 7) | |
| | Total | 3 |

Blocking of lysines and arginines (by reductive methylation and reaction with cyclohexadione, respectively) followed by tryptic digestion and analysis via LC-MS results in the identification of only 2 peptides in a total of 3 spectra (Table 2), indicating that streptavidin has become refractory to digestion with trypsin (see also FIG. 1C).

### Example 3

### Peptides Identified from Streptavidin before and after Chemical Blocking of Lysines and Enzymatic Conversion of Arginine to Citrulline

The enzyme Peptidylarginine deiminase (PAD) converts Arginine into citrulline (FIG. 4), which is not a substrate of trypsin thus resulting in resistance to proteolytic cleavage.

This was confirmed in an experiment where Lysines in streptavidin were blocked chemically (as in example 2) followed by enzymatic treatment with PAD to convert arginines into citrulline. Tryptic digestion led to the identification of 2 peptides in a total of 7 spectra (Table 3) which is in stark contrast to the 411 spectra observed after digestion of unmodified trypsin (Table 1), and indicating a similar level of protease resistance as chemically modified streptavidin (Table 2). Of note, detection of the arginine-flanked peptide

R.NAHSATTWSGQYVGGAEAR.I (SEQ ID NO: 3) was reduced from 88 from spectra in unmodified streptavidin (Table 1) to 6 spectra after PAD treatment (Table 3). Collectively, this shows that both chemical and enzymatic derivatization of Arginine leads to resistance to proteolysis by trypsin.

TABLE 3
Tryptic peptides generated from streptavidin
after chemical blocking of K's and enzymatic
conversion of R's by Peptidylarginine
deiminase (PAD)

| | | number |
|---|---|---|
| mass | | of |
| (Da) | Sequence | spectra |
| 1962.91 | R.NAHSATTWSGQYVGGAEAR.I | 6 |
| | (SEQ ID NO: 3) | |
| 2154.01 | R.INTQWLLTSGTTEANAWK.S | 1 |
| | (SEQ ID NO: 4) | |
| | Total | 7 |

From the sequences of the peptides obtained before blocking and after blocking of lysine and arginine residues it can be concluded that some residues are more critical than others in order to render streptavidin resistant to cleavage by trypsin. In particular, blocking R59, R84, R103, K121, and K132 of SEQ ID NO: 2 (corresponding to R83, R108, R127, K145, and K156, respectively, of SEQ ID NO: 1) will render streptavidin resistant to cleavage by trypsin. These five critical amino acid residues are marked by underlining in the following sequence:

(SEQ ID NO: 2)
DPSKDSKAQV SAAEAGITGT WYNQLGSTFI VTAGADGALT
GTYESAVGNA ESRYVLTGRY DSAPATDGSG TALGWTVAWK
NNYRNAHSAT TWSGQYVGGA EARINTQWLL TSGTTEANAW
KSTLVGHDTF TKVKPSAASI DAAKKAGVNN GNPLDAVQQ

It is assumed that other arginine and lysine residues are not accessible by trypsin, or at least cleavage seems to be less efficient.

### Example 4

### Capture and Identification of the PRC2-Complex Bound to Biotinylated DNA from a Chromatin Sample via LysC-Resistant Streptavidin Beads

Experimental set up: from formaldehyde-crosslinked and sheared chromatin a Chromatin immuno-precipitation (ChIP) experiment was performed using an antibody against Suz12, one of the core components of the PRC2-complex. Next, DNA was biotinylated in the presence of biotinylated nucleotides by a DNA polymerase, and the DNA (along with proteins cross-linked to it) was captured on streptavidin beads (either using unmodified streptavidin or

LysC-resistant streptavidin achieved by blocking lysines by reductive methylation). After extensive washing, proteins were digested with LysC, peptides were collected and identified by mass spectrometry.

A schematic representation of the PRC2-complex is shown in FIG. 2A (Symbols: Triangle: Suz12; open ovals: proteins in the PRC2 complex; grey ovals: other transiently associated proteins; black line: DNA; solid black circle: biotin on DNA; inverted Y: Suz12 antibody; inverted C: streptavidin (either unmodified or after blocking of Lysines)).

Proteins identified by mass spectrometry after the above-described procedure are listed in the table shown in FIG. 2B. Identified proteins are indicated by gene name; those known to belong to the PRC2 complex are indicated in bold italics. The column with the header “Peptides” lists the number of peptides identified per protein. The column with the header “PSM” (i.e. peptide-spectrum matches) indicates the number of times these peptides were identified per protein.

Note that when using normal (unmodified) streptavidin, 6 streptavidin peptides were identified a total of 637 times, indicating their high abundance. In contrast, after blocking lysines only 1 streptavidin peptide was identified only 2 times, indicating very low abundance. All other proteins, including the entire PRC2 complex, were identified with a highly comparable number of peptides and PSM both on unmodified and Lysine-blocked streptavidin. Importantly, the high abundance of streptavidin peptides in the sample from normal streptavidin necessitated peptide fractionation into 10 fractions to reduce complexity and to facilitate detection of peptides that would otherwise be masked by streptavidin, requiring 10 LC-MS runs. In contrast, the sample generated from the LysC-resistant streptavidin was analysed without fractionation in a single LC-MS run. Therefore, the results from the LysC-resistant beads required only 10% of the mass spectrometry time, while essentially identifying the same proteins with the same number of peptides compared to the sample from normal streptavidin.

### Example 5

### Modification of Lysines and Arginines Minimally Affects Binding Capacity

The binding capacity of beads coated with different types of streptavidin was assessed by determining the recovery of biotinylated DNA (FIG. 3).

The binding capacity of K&R-modified streptavidin beads is maintained at 75% (middle column) compared to normal streptavidin (left column), while binding capacity after K-modification is even increased by about 30% (right column).

### Example 6

### Capture and Identification of the PRC2-Complex Bound to Biotinylated DNA from a Chromatin Sample via Trypsin-Resistant Streptavidin Beads

Experimental set up: The experimental set up was the same as in Example 4 with the exception that trypsin-resistant streptavidin was used instead of LysC-resistant streptavidin. Trypsin-resistant streptavidin was prepared by blocking lysines by reductive methylation and by blocking arginines by reaction with cyclohexadione.

A schematic representation of the PRC2-complex is shown in FIG. 2A (Symbols: Triangle: Suz12; open ovals: proteins in the PRC2 complex; grey ovals: other transiently associated proteins; black line: DNA; solid black circle: biotin on DNA; inverted Y: Suz12 antibody; inverted C: streptavidin (either unmodified or after blocking of Lysines and Arginines)).

After capture, elution and digestion of the DNA-bound PRC2 complex on regular streptavidin beads, the LC-MS chromatogram is dominated by streptavidin-peptides (FIG. 5, upper diagram), which is in contrast to capture on K&R-modified streptavidin (FIG. 5, lower diagram). Note the different intensity scales (10⁹ vs. 10⁷).

The number of peptide-spectrum matches (PSMs) in the analysis of the PRC2 complex enriched on regular streptavidin beads and on K&R-modified streptavidin beads is shown in the table presented in FIG. 6. The column entitled “regular streptavidin” corresponds to the upper diagram of FIG. 5, and the column entitled “K/R-modified streptavidin” corresponds to the lower diagram of FIG. 5.In the absence of peptides from modified streptavidin, each of the core components of thePRC2-complex was identified by a larger number of PSMs when using K&R-modified beads. In addition, 224 other proteins were identified, compared to only 78 when using regular streptavidin.

The overall gain in sensitivity afforded by K&R-modified streptavidin (see FIG. 5) is the result of the consistent higher ion intensity for all proteins (FIG. 7A), and a larger number of PSMs for each of them (FIG. 7B).

### SEQUENCE LISTING FREE TEXT INFORMATION

SEQ ID NO: 3 tryptic fragment of streptavidin
SEQ ID NO: 4 tryptic fragment of streptavidin
SEQ ID NO: 5 tryptic fragment of streptavidin
SEQ ID NO: 6 tryptic fragment of streptavidin
SEQ ID NO: 7 tryptic fragment of streptavidin
SEQ ID NO: 8 tryptic fragment of streptavidin
SEQ ID NO: 9 tryptic fragment of streptavidin
SEQ ID NO: 10 tryptic fragment of streptavidin
SEQ ID NO: 11 tryptic fragment of streptavidin
SEQ ID NO: 12 tryptic fragment of streptavidin

1. A modified streptavidin that
(i) is resistant to cleavage by at least one endopeptidase, wherein said at least one endopeptidase is specific for a basic amino acid; and
(ii) exhibits a dissociation constant K_{D} to biotin of 10¹⁰ M or less;
wherein said at least one endopeptidase is preferably selected from the group consisting of LysC, LysN, ArgC, and trypsin.

2. The modified streptavidin according to claim 1, wherein said at least one endopeptidase is selected from the group consisting of LysC, LysN, and trypsin, and wherein one or more lysine residues carry at least one chemical modification selected from the group consisting of:
a chemical modification that neutralizes the positive charge of the c amino group; and
(ii) a chemical modification that replaces a hydrogen of the c amino group.

3. The modified streptavidin according to claim 2, wherein said chemical modification is produced by a chemical reaction selected from the group consisting of:
(i) acylation of lysine residues producing acyl-lysine;
(ii) reductive alkylation of lysine residues producing dialkyl-lysine;
(iii) reaction of lysine residues with propionic anhydride producing propionyl lysine;
(iv) reaction of lysine residues with succinic anhydride producing lysine dicarboxylic anhydride;
(v) alkylation of lysine residues producing alkyl-lysine;
(vi) amidination of lysine residues producing the acetimidine derivative of lysine;

4. The modified streptavidin according to claim 1, wherein said at least one endopeptidase is selected from the group consisting of LysC, LysN, and trypsin, and wherein said modified streptavidin is a mutein of a wild-type streptavidin amino acid sequence according to SEQ ID NO: 2, wherein said mutein is characterized by amino acid exchanges at least in positions K121 and K132 of SEQ ID NO: 2, and wherein said mutein comprises one or more of: between 1 and 10 internal amino acid deletions, between 1 and 10 amino acid insertions, between 1 and 10 amino acid exchanges, between 1 and 13 N-terminal deletions, and/or between 1 and 20 C-terminal deletions.

5. The modified streptavidin according to claim 4, wherein K121 and K132 have been replaced, independently from each other, by another amino acid, wherein said another amino acid is neither lysine nor arginine; and
wherein the amino acids replacing K121 or K132 are, independently from each other, selected from the group consisting of alanine, asparagine, aspartic acid, cysteine, glutamic acid, glutamine, glycine, histidine, isoleucine, leucine, methionine, phenylalanine, proline, serine, threonine, tryptophan, tyrosine, valine, hydroxy-proline, acetyl-ornithine, acetamido-methyl-cysteine, O-acetamido-methyl-homo-serine, S-acetamido-methyl-homo-cysteine, acetyl-lysine, propionyl-lysine, hydroxyl-acetyl-lysine, monofluoroacetyl-lysine, difluoroacetyl-lysine, trifluoroacetyl-lysine, crotonyl-lysine, and dimethyl-lysine.

6. The modified streptavidin according to claim 1, wherein said at least one endopeptidase is selected from the group consisting of ArgC and trypsin, and wherein one or more arginine residues carry at least one chemical modification selected from the group consisting of:
(i) a chemical modification that neutralizes the positive charge of thea guanidinium group of the one or more arginine residues; and
(ii) a chemical modification that replaces one or more hydrogens of the guanidinium group of the one or more arginine residues.

7. The modified streptavidin according to claim 6, wherein said chemical modification is produced by a chemical reaction selected from the group consisting of:
(i) reaction of arginine residues with dicarbonyl compounds producing a modified arginine residue;
(ii) carbamylation of arginine residues producing carbamylated arginine: and
(iii) de-imination of arginine residues producing citrulline residues.

8. The modified streptavidin according to claim 1, wherein said at least one endopeptidase is selected from the group consisting of ArgC and trypsin, and wherein said modified streptavidin is a mutein of a wild-type streptavidin amino acid sequence according to SEQ ID NO: 2, wherein said mutein is characterized by one or more amino acid exchanges in positions R59, R84, or R103 of SEQ ID NO: 2, and wherein said mutein comprises one or more of: between 1 and 10 internal amino acid deletions, between 1 and 10 amino acid insertions, between 1 and 10 amino acid exchanges, between 1 and 13 N-terminal deletions, and/or between 1 and 20 C-terminal deletions.

9. The modified streptavidin according to claim 8, wherein R59, R84, or R103 have been replaced, independently from each other, by another amino acid, wherein said another amino acid is neither lysine nor arginine; and
wherein the amino acids replacing R59, R84, or R103 are, independently from each other, selected from the group consisting of alanine, asparagine, aspartic acid, cysteine, glutamic acid, glutamine, glycine, histidine, isoleucine, leucine, methionine, phenylalanine, proline, serine, threonine, tryptophan, tyrosine, valine, hydroxy-proline, acetyl-ornithine, acetamido-methyl-cysteine, O-acetamido-methyl-homo-serine, S-acetamido-methyl-homo-cysteine, acetyl-lysine, propionyl-lysine, hydroxyl-acetyl-lysine, monofluoroacetyl-lysine, difluoroacetyl-lysine, trifluoroacetyl-lysine, crotonyl-lysine, and dimethyl-lysine.

10. A solid support comprising the modified streptavidin of claims 1, wherein the solid support is selected from the group consisting of beads, tubes, chips, resins, plates, wells, films, sticks, magnetic beads, porous membranes and combinations thereof.

11. A kit comprising the modified streptavidin of claim 1, further comprising at least one protease selected from the group consisting of LysC, LysN, ArgC, and trypsin.

12. A use of the modified streptavidin of claim 1, for capture or immobilization of at least one biotinylated molecule; wherein the at least one biotinylated molecule is selected from the group consisting of proteins, peptides, oligonucleotides, polynucleotides, lipids, (poly)saccharides, carbohydrates, metabolites, drugs, small molecules, natural molecules, and synthetic molecules.

13. A use of the modified streptavidin of claim 1, in mass spectrometry, for reducing background in mass spectrometry.

14. A method for reducing background in mass spectrometry, comprising the steps:
(i) providing beads carrying the modified streptavidin according to claim 1;
(ii) contacting a sample comprising a biotinylated protein with the beads of step (i), thereby binding the biotinylated protein to the modified streptavidin;
(iii) optionally washing the beads with a wash buffer;
(iv) adding a solution comprising a protease, preferably a protease selected from the group consisting of LysC, LysN, ArgC, and trypsin, to the beads, thereby generating peptide fragments of the biotinylated protein;
(v) recovering the peptide fragments generated in step (iv); and
(vi) optionally subjecting the peptide fragments recovered in step (v) to mass spectroscopic analysis.

15. A method for capturing a protein interaction partner of a molecule, comprising the steps:
(i) providing beads carrying the modified streptavidin according to claim 1;
(ii) contacting a biotinylated molecule with the beads of step (i), thereby loading the biotinylated molecule onto the beads, wherein preferably the biotinylated molecule is selected from the group consisting of proteins, peptides, oligonucleotides, polynucleotides, lipids, (poly)saccharides, carbohydrates, metabolites, drugs, small molecules, natural molecules, and synthetic molecules;
(iii) contacting a sample with the beads loaded with the biotinylated molecule obtained in step (ii), wherein said sample comprises at least one protein interaction partner for the biotinylated molecule;
(iv) optionally washing the beads with a wash buffer;
(v) adding a solution comprising a protease selected from the group consisting of LysC, LysN, ArgC, and trypsin, to the beads, thereby generating peptide fragments of the at least one protein interaction partner;
(vi) recovering the peptide fragments generated in step (v); and
(vii) optionally subjecting the peptide fragments recovered in step (v) to mass spectroscopic analysis.


SEQUENCE LISTING (13 sequences)

SEQ ID NO: 1 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 159 aa, Streptomyces avidinii
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 3 - protein, 21 aa, Artificial Sequence
  Tryptic fragment of streptavidin
RNAHSATTWSGQYVGGAEARI

SEQ ID NO: 4 - protein, 20 aa, Artificial Sequence
  Tryptic fragment of streptavidin
RINTQWLLTSGTTEANAWKS

SEQ ID NO: 5 - protein, 13 aa, Artificial Sequence
  Tryptic fragment of streptavidin
KSTLVGHDTFTKV

SEQ ID NO: 6 - protein, 14 aa, Artificial Sequence
  Tryptic fragment of streptavidin
KVKPSAASIDAAKK

SEQ ID NO: 7 - protein, 23 aa, Artificial Sequence
  Tryptic fragment of streptavidin
RYDSAPATDGSGTALGWTVAWKN

SEQ ID NO: 8 - protein, 25 aa, Artificial Sequence
  Tryptic fragment of streptavidin
KNNYRNAHSATTWSGQYVGGAEARI

SEQ ID NO: 9 - protein, 29 aa, Artificial Sequence
  Tryptic fragment of streptavidin
RYVLTGRYDSAPATDGSGTALGWTVAWKN

SEQ ID NO: 10 - protein, 31 aa, Artificial Sequence
  Tryptic fragment of streptavidin
RINTQWLLTSGTTEANAWKSTLVGHDTFTKV

SEQ ID NO: 11 - protein, 19 aa, Artificial Sequence
  Tryptic fragment of streptavidin
NAHSATTWSGQYVGGAEAR

SEQ ID NO: 12 - protein, 18 aa, Artificial Sequence
  Tryptic fragment of streptavidin
INTQWLLTSGTTEANAWK

SEQ ID NO: 13 - protein, 11 aa, Artificial Sequence
  Tryptic fragment of streptavidin
STLVGHDTFTK